(19) United States
(12) Patent Application Publication
(10) Pub. No.: US 2019/0151503 A1
(43) Pub. Date: May 23, 2019

(54) COATING FOR IMPROVED TISSUE ADHESION

(71) Applicant: ID Creations Oy, Turku (FI)

(72) Inventors: Illka KANGASNIEMI, Piispanristi (FI); J. Pablo PEREZ, Bilbao (ES); Natasa DRNOVSEK, Ljubljana (SI); Martina LORENZETTI, Colle di Val d'Elsa (IT)

(73) Assignee: ID Creations Oy, Turku (FI)

(21) Appl. No.: 16/085,556

(22) PCT Filed: Mar. 15, 2017
(86) PCT No.: PCT/FI2017/050170

§ 371 Date: Sep. 14, 2018

(30) Foreign Application Priority Data
Mar. 16, 2016 (FI) 20165218

(51) Int. Cl. A61L 27/30; A61L 27/36; A61L 27/54

(52) U.S. Cl. CPC A61L 27/306; A61L 27/06; A61L 27/54; A61L 27/3691

(57) ABSTRACT

The invention relates to a coating consisting essentially of titanium dioxide wherein at least 20% of the titanium dioxide has a crystalline structure of anatase and/or rutile; the coating has a roughness comprising indentations, wherein at least 50% of the indentations have a maximum depth of 1-50 nm and a maximum width of 1-50 nm; the coating is treatable to achieve a water contact angle of 0-20°; the coating is treatable to be negatively charged or positively charged; and the coating exhibits an improved attachment of mammalian tissue cells, which improved attachment is such that when a substrate coated with the above coating is compared to the same uncoated substrate, at least 100% more cells remain attached on the coated substrate than on the uncoated substrate.

### FIELD OF THE INVENTION

The present invention relates to a coating consisting essentially of titanium dioxide. The invention also relates to use of the coating, to various devices coated with the present coating as well as to a method for manufacturing the coating.

### BACKGROUND OF THE INVENTION

Titanium dioxide is known to be used for coating medical implants that are to be attached to bone. Titanium dioxide has typically a partially or fully crystalline structure in anatase and/or rutile form and reasonably good results have been achieved. According to some studies, treatment of such a surface by UV-light or argon ion plasma treatment (hereinafter Ar ion plasma) cleans the surface of carbohydrates and improves the hydrophilicity of the surface and may even render it superhydrophilic, which in turn improves attachment, adhesion and proliferation of bone and other tissue cells to the surface.

It is known from the studies with photocatalysis of TiO₂ that the surfaces of anatase and rutile surfaces can be made superhydrofilic and clean by using UV-light excitation. It has also been shown that the clean surface improves the protein adsorption and cell attachment leading to improved bone tissue growth and integration of the implant.

In other studies, it has been shown that the surface structure has a significant effect on cell and tissue adhesion also with soft tissues. In publication WO 02/87648 it has been explained how surface roughness below 50 nm enables an improved adhesion strength of soft tissue cells and tissues on the implant surface. In animal experiments, it was observed that often the adhesion of soft tissue to the implant was even greater than that of the internal strength of the tissue itself, leading to a cohesive rupture of the tissue in a mechanical pull out test. The same phenomenon has been observed also in clinical cases recently.

It is to be noted that cell attachment is not equivalent to cell adhesion. Indeed, attachment refers to good contact, spreading and biocompatibility of cells on a surface. However, such cells are often also easily detached. “Attachment” in literature never refers to strength of adhesion. Furthermore, for bone applications, cell adhesion strength is irrelevant as the mineralized bone anchoring the implant creates a situation where the cell and tissue adhesion is completely overshadowed by it.

Various medical implants and other devices are arranged in connection with a patient's soft tissues. Some examples are for example catheters and stents. For catheters, that are arranged to pass through skin and to remain both outside the body and inside, the wound made for inserting the catheter is prone to infections as the skin cells do not adhere well to the outer surface of the catheter. The wound remains open and inflammated as a result of foreign body reaction as well as continuous bacterial attack from outside the wound. A very similar situation occurs in a number of indications e.g. for orthopaedic fixation pins, dental implants, maxillofacial prosthetics fixation screws.

For stents arranged in a vein of a patient, non-adhesion of the stent may result in blood by-passing the stent, instead of flowing through the stent. The stent may also move as a result of non-adhesion and non-integration.

An adhesion in itself is often a desirable outcome, because it can directly prevent complications such as described above. The adhesion also results in other types of benefits that are indirect. It is shown in our own previous studies that the adhered tissue does not elicit a foreign body reaction leading to a chronic inflammatory response and encapsulation of the implant. For dental implants, as a result of the lesser inflammatory response, the bone resorption underneath the soft tissue adhered gingival area has been shown to reduce. Inflammatory signals of tissue are known to affect also bone tissue healing around dental implants.

There exists thus a need to provide a manner for improving adhesion of various medical devices to soft tissues of humans and animals.

### OBJECTS AND SUMMARY OF THE INVENTION

It is thus an object of the present invention to provide a coating that enhances soft tissue adhesion to the coated surface. It is another object to provide medical devices capable of soft tissue adhesion. A further object is to provide a method for coating devices for obtaining good soft tissue adhesion properties. A further object is to provide a coating having good soft tissue adhesion properties and also good storability. A yet further object is to provide a coating that at least partially overcomes the problems of prior known coatings and surface treatments.

The present description relates to a coating consisting essentially of titanium dioxide. In a typical coating,
at least 20% of the titanium dioxide has a crystalline structure of anatase and/or rutile,
the coating has a surface comprising indentations, wherein at least 50% of the indentations have a maximum depth of 1-50 nm and a maximum width of 1-50 nm,
the coating is treated to achieve a water contact angle of 0-20°, and
the treated coating exhibits an attachment of mammalian tissue cells capable of adhesion, which attachment is such that when a substrate coated with the above coating and treated is compared to the same uncoated and untreated substrate, at least 100% more cells remain attached on the coated, treated substrate than on the uncoated, untreated substrate, the attachment of cells being measured by a method in which
   six parallel plate-like samples of both coated, treated substrate and uncoated, uncoated substrate having a largest diameter of 8 mm and a thickness of 0.1-1 mm are used,
   the samples are stored in an aqueous solution for at least 20 hours prior to the measurement,
   soft tissue cells are cultured on the coated, treated substrate samples and uncoated, untreated substrate samples for 6 hours at 37° C. in a 5% carbon dioxide atmosphere,
   the cultured coated, treated substrate samples and the cultured uncoated, untreated substrate samples are subjected to trypsinisation with a solution of trypsin in phosphate buffered saline in an orbital shaker at 50 rpm for 12 min at room temperature, and
   remaining attached cells are counted from the samples and the average of the six parallel samples is calculated, wherein the volume-% of trypsin in the solution of trypsin is selected such that on average at least 300 cells/cm² remain attached on the uncoated, untreated substrate samples.

The present description also relates to use of a coating as described above, for Improving soft tissue cell adhesion of a device. The description also relates to a medical or cosmetic device intended to penetrate a skin or to be implanted into a mammal, comprising a coating as described above.

The description still further relates to a method for manufacturing a device capable of soft tissue adhesion. A typical method comprises the steps of
manufacturing a coating layer consisting essentially of titanium dioxide wherein
   at least 20% of the titanium dioxide has a crystalline structure of anatase and/or rutile, and
   the coating has a surface comprising indentations, wherein at least 50% of the indentations have a maximum depth of 1-50 nm and a maximum width of 1-50 nm, and
treating the coating to achieve a water contact angle of 0-20°.

### BRIEF DESCRIPTION OF THE DRAWING

FIGS. 1-7 illustrate some results of in vitro tests.

FIG. 8 is a microscopic image of a tested surface.

FIGS. 9A-9C illustrate results of elemental analysis of the surface shown in FIG. 8.

### DETAILED DESCRIPTION OF THE INVENTION

The present description relates to a coating consisting essentially of titanium dioxide. In a typical coating,
at least 20% of the titanium dioxide has a crystalline structure of anatase and/or rutile,
the coating has a surface comprising indentations, wherein at least 50% of the indentations have a maximum depth of 1-50 nm and a maximum width of 1-50 nm,
the coating is treated to achieve a water contact angle of 0-20°, and
the treated coating exhibits an attachment of mammalian tissue cells capable of adhesion, which attachment is such that when a substrate coated with the above coating and treated is compared to the same uncoated and untreated substrate, at least 100% more cells remain attached on the coated, treated substrate than on the uncoated, untreated substrate, the attachment of cells being measured by a method in which
   six parallel plate-like samples of both coated, treated substrate and uncoated, uncoated substrate having a largest diameter of 8 mm and a thickness of 0.1-1 mm are used,
   the samples are stored in an aqueous solution for at least 20 hours prior to the measurement,
   soft tissue cells are cultured on the coated, treated substrate samples and uncoated, untreated substrate samples for 6 hours at 37° C. in a 5% carbon dioxide atmosphere,
   the cultured coated, treated substrate samples and the cultured uncoated, untreated substrate samples are subjected to trypsinisation with a solution of trypsin in phosphate buffered saline in an orbital shaker at 50 rpm for 12 min at room temperature, and
   remaining attached cells are counted from the samples and the average of the six parallel samples is calculated, wherein the volume-% of trypsin in the solution of trypsin is selected such that on average at least 300 cells/cm² remain attached on the uncoated, untreated substrate samples.

The present coating has several advantageous effects when used in devices implanted into a mammalian (human or animal) body. Indeed, in contact with a fresh wound it improves cell and tissue adhesion to higher level than either a nanostructured TiO₂ surface or a UV- or plasma functionalised TiO₂ or anatase or rutile crystalline surface, as known in the art. The present coating thus allows better results than with coatings known in the art. The present inventors believe, without however wishing to be bound by a theory that this advantageous effect is obtained with the specific combination of the crystalline structure and surface roughness. Indeed, even though with bone cells, bone growth has been shown to improve on surfaces having indentations deeper than 50 nm, the improved cell adhesion with soft tissue cells has only been observed with surfaces where the indentation are at most 50 nm in depth.

A further advantage is that when the device is skin-penetrating and remains both inside the body and outside it, it improves the healing process of the wound surrounding the device, due to the improved soft tissue adhesion. It thus also improves the closing of the wound and thus reduces the risk of infections as no bacteria can enter the wound once it is closed. The present coating also has the advantageous effect of reducing bone resorption next to the coating. The good soft tissue adhesion properties of the present coating has the yet further effects of reducing the probability of peri-implant infections, reducing the probability of dehiscence and reducing the probability of movement of the implant due to detachment.

Indeed, when a coating according to the present description has been manufactured and tested for soft tissue adhesion clinically cohesive rupture of tissue surrounding the treated surface (i.e. the coated surface, i.e. the coating) has been observed. The surface adhesion of the tissue is thus higher than the strength of the tissue itself. This will be explained in more detail below in the Experimental part. When the terminology of “improved attachment” is used, it is meant that more cells attached to the surface than to an uncoated substrate.

Cells that are capable of adhesion are to be understood to be cells like fibroblasts, endothelial cells, epithelial cells, chondroblasts etc, and exclude cells that are by nature not able to adhere, like blood cells, white cells etc. Some cells are not capable of adhesion in the same meaning as for instance fibroblasts, like nerve cells, and in that case the measurement according to the present description is not possible to be performed. However the present coating may improve adhesion of such cells also, although it is most probably not possible to test it according to the present method.

One characteristic of the coating is that when soft tissue cells are cultured on the coating for 6 hours at 37° C. in a 5% carbon dioxide atmosphere, they adhere to the coating. The adhesion can be measured by trypsinisation. Indeed, trypsinisation with a solution of trypsin in phosphate buffered saline, in an orbital shaker at 50 rpm for 12 min at room temperature detach some of the cells attached to the surface by the cell culture. The amount of cells that remain attached (i.e. that do not detach) is higher with the present coating, when compared to an uncoated substrate. Furthermore, the remaining cell count is at least 45% higher than on a treated metallic titanium surface and at least 30% higher than on an untreated titanium dioxide coated surface having the same crystalline structure and roughness. The good adhesion is thus obtained with the combination of the crystalline structure, the surface roughness and the treatment of such surface. This method of trypsinisation is known per se to a person skilled in the art, but is also explained in more detail in the Experimental part below.

Due to the fact that cell types and lines are different, the amount of trypsin in the trypsination step needs to be set at an appropriate level in order to be able to observe the difference. Indeed, the present inventors have observed that the amount of cells that seem to be attached on the surface in 6 h is approximately the same with both coated and uncoated substrates, but the attachment force varies, hence the trypsination detaches more cells from the uncoated surfaces than it does from the coated surfaces, when the conditions are otherwise identical. The amount of trypsin (in volume-% in the solution in phosphate buffered saline) is selected such that at least 300 but not more than 2000 cells/cm² remain attached on the uncoated substrate samples. Indeed, if the amount of cells that remain attached on the uncoated substrate samples is too high (say 80% of the untrypsinated cell amount, which typically can be 6000-8000 cells/cm²), the difference between the uncoated sample and coated sample (at least 100%) is too low for statistical analysis. If the cell count drops close to 0 on the noncoated trypsinated group, then it is already unclear if there is a measurable adhesion of the cells observable and again, a comparison may be impossible. A person skilled in the art is readily able to determine the correct amount of trypsin to be used, with a few simple trial-and-error experiments. Preferably, the amount of trypsin is selected such that about 1000-2000 cells/cm² remain attached on the uncoated substrate samples, in order to facilitate the counting of the cells. Indeed, if the amount is too high, counting the cells that remain attached may be too cumbersome and not lead to verifiable results.

It is also to be noted that direct comparison between different materials is typically not very reliable, as it is highly difficult to make exactly identical test samples from various materials to be coated. Moreover, when the coating according to the present disclosure is tested, substantially the whole surface of the substrate is coated. In case only a part of the substrate is coated, then the comparison is made between a portion of the substrate that is coated and an uncoated substrate

Indeed, the prior art does not teach the combination of a surface structure having indentations with depth and width below 50 nm, the crystallinity of TiO₂ and cleaning and or excitation of the surface with UV-light, Ar ion plasma or hydrogen peroxide in order to achieve a high soft tissue adhesion strength. While it is know that cells and tissues have been able to attach and adhere to the kinds of surfaces described earlier, none of these surface qualities and treatments alone have achieved as high tissue adhesion as the combination. The improvement of greater adhesion leads to more repeatable clinical outcomes and better resistance against mechanical trauma of the interface, thus leading to significant improvements in treatment results as well as decreased costs for the treatment. Indeed, when the device (for example a catheter or a dental implant) adheres well to the soft tissue, the bacteria cannot enter the wound and the device also does not easily move, thus improving the healing process in general.

According to an embodiment, the aqueous solution used in the measurement method is selected from deionised water and an aqueous solution comprising serum. The serum can be for example DMEM (Dulbecco's Modified Eagle's Medium), which is a 10% diluted serum, containing thus a fairly low concentration of proteins. The solution used in the measurement method may of course also be an undiluted serum.

When the coating is manufactured, the last treatment step may be carried out immediately before use of the coated device (i.e. before its implantation into mammalian tissue). In this case, no storage of the coated device is needed as the coated device is immediately brought into contact with tissue fluids and blood, which thus start reacting with the surface as soon as the contact is formed. According to an embodiment, the coating is treatable to be negatively charged by cleaning of naturally adsorbed hydrocarbons by hydrogenperoxide or positively charged by cleaning the carbohydrates and photocatalytically exciting the surface with UV light under 360 nm or by argon ion plasma, while at the same time achieving a water contact angle of 0-200.

According to another embodiment, the manufacturing process of the coating comprises a step of decreasing the water contact angle by at least one of
cleaning the surface of naturally adsorbed hydrocarbons by hydrogen peroxide or calcium peroxide, or
cleaning the carbohydrates and photocatalytically exciting the surface with UV light under 360 nm or by argon ion plasma or by oxygen plasma, or by adsorbing calcium on the surface.

Indeed, according to the present disclosure, the low water contact angle of the coating is achieved by a treatment of the coating itself, and not by adding an additional layer such as a top coat on it.

The amount of cells remain attached on the coated substrate when compared to the uncoated substrate is at least 100% more, preferably at least 1200% more and more preferably at least 150% more. The amount can be for example at least 100, 105, 110, 115, 120, 125, 130, 135, 140, 145, 150, 155, 160 or 165% more. The amount can of course also be significantly more, such as 200 or 250% more, sometimes even 300% or more. The amount is determined by any suitable manner, one possibility being by imaging and calculation. This method is explained in more detail below in the Experimental part.

According to an embodiment, the coating is treated to be negatively charged or positively charged. The present coating may thus be treatable to be negatively charged, for example by cleaning of naturally adsorbed hydrocarbons by (diluted) hydrogen peroxide or positively charged for example by cleaning the carbohydrates and photocatalytically exciting the surface with UV light under 360 nm or Ar ion plasma, to achieve a water contact angle of 0-20°. Indeed, there are various methods for achieving the very low water contact angle. One method is to clean the surface of the coating of naturally adsorbed hydrocarbons, which makes the coating negatively charged. The cleaning may be made for example with hydrogen peroxide or hydrogen peroxide plasma. The treatment with hydrogen peroxide can be carried out for example by immersing the surface to be cleaned in liquid hydrogen peroxide or by subjecting the surface to be cleaned to oxygen radicals that are released from liquid hydrogen peroxide. It is also believed that calcium peroxide could also be used and that it would act like hydrogen peroxide, i.e. cleaning the surface of carbohydrates, but releasing also Ca ions into the solution, leading to immediate adsorption of the ions by the negatively charged TiO₂ surface. The surface could also be treated, directly after its manufacturing, with Ca ions.

The surface of the coating may also be rendered positively charged by photocatalytically exciting it with ultraviolet light having a wavelength under 360 nm or by Ar ion plasma or by oxygen plasma. Thus, the coating has a high hydrophilicity (low water contact angle), but it is also charged, either positively or negatively. It is believed that the charge of the surface, i.e. the fact that it is electrostatic, improves further the adhesion of the soft tissue cells to the surface. It is furthermore believed that when the surface is negatively charged, it will be able to adsorb Ca²⁺ ions from the interstitial fluid. This adsorption makes the surface positive, which then in turn allows for proteins to be adsorbed on the surface. On the other hand, when the surface is positively charged, it will be able to adsorb proteins directly.

The coating can be manufactured in any suitable manner allowing the formation of the specific crystalline structure and surface roughness. Some examples of manufacturing methods are a sol-gel process, a hydrothermal process, a pulsed laser deposition process, an ion implantation process, an electrospraying process, a chemical vapour deposition process, a physical vapour deposition process, a laser beam machining process and a three dimensional growing process.

The coating is hydrophilizable to achieve a water contact angle of 0-20°, as mentioned above. This low water contact angle can be achieved either directly by the manufacturing method or the manufacturing process may comprise a step of decreasing the water contact angle by any of the methods mentioned above. There may be also other ways of decreasing the water contact angle down to a maximum of 20°.

According to an embodiment, the water contact angle is 0-10°. The water contact angle can also be from 0, 1, 2, 3, 4, 5, 6, 7, 8, 9, 10, 11, 12, 13, 14, 15, 16, 17 or 180 up to 1, 2, 3, 4, 5, 6, 7, 8, 9, 10, 11, 12, 13, 14, 15, 16, 17, 18, 19 or 20°. Typically, the lower the water contact angle, the better.

The coating has a surface comprising indentations, wherein at least 50% of the indentations have a maximum depth of 1-50 nm and a maximum width of 1-50 nm, i.e. the surface has a certain roughness. The surface roughness can be measured by any suitable means, for example by high resolution scanning electron microscope (SEM) or atomic force microscopy (AFM). The depth is measured from bottom of an indentation to an adjacent peak and the width is measured from peak-to-peak. The indentations are to be understood to comprise also the situation where the surface is made of particles or crystals. In this case, the particles or crystals are attached to each other as clusters in such a manner that the clusters are between 1-50 nm in width and height, and there are parts of the surface not comprising such clusters (or clusters that are located lower than the outermost surface).

According to an embodiment, at least 60% of the indentations have a maximum depth of 1-50 nm and a maximum width of 1-50 nm. According to a further embodiment, the percentage of the indentations having the defined size (depth and width) is from 50, 55, 60, 65, 70 or 75% up to 60, 65, 70, 75, 80, 85, 90 or 95%. According to yet another embodiment, the maximum depth of the indentations is from 1, 2, 5, 7, 10, 15, 20, 25, 30, 35, 40 or 45 nm up to 2, 5, 7, 10, 15, 20, 25, 30, 35, 40, 45 or 50 nm. According to a further embodiment, the maximum width of the indentations is from 1, 2, 5, 7, 10, 15, 20, 25, 30, 35, 40, or 45 nm up to 1, 2, 5, 7, 10, 15, 20, 25, 30, 35, 40, 45 or 50 nm. The width and depth of the indentations can be selected independently from each other, as well as can the percentage of the indentations having these specific dimensions.

The coating can be stored in an aqueous solution. It has indeed been noticed that the advantageous properties can be retained by storing the coated device in an aqueous solution. Should storage in aqueous solution not be possible, the coating can be re-activated (if need be) by subjecting it again to ultraviolet light excitation, argon ion plasma excitation, oxygen plasma excitation or white light excitation, or any other suitable manner for reducing the water contact angle.

The present coating can be used on a number of materials. Some suitable materials (i.e. the substrate in the testing method) can be selected from a group consisting of titanium, a titanium alloy, tantalum, niobium, an alloy of cobalt and chromium, steel, zirconium, aluminium oxide, titanium nitride, zirconium nitride, silica-glass, quartz, a polymer and a polymer composite. Concerning the polymer composites, a component of the composite can be TiO₂.

The present description also relates to use of a coating according to this description, for improving mammalian tissue cell adhesion of a device. The description further relates to a medical or cosmetic device intended to penetrate a skin or to be implanted into a mammal, comprising a coating according to the present description.

According to an embodiment, the device is selected from a group consisting of orthopaedic fixation pins, craniomaxillofacial prosthesis fixation screws, catheters, cannulae, vascular stents, aneurysm stents, annular rings for heart valves, removable prosthesis implants for extremities, abdominal catheters, urethral catheters, esophageal stents, dental implant abutments, tissue level implants abutment sections and skin penetrating jewelry. The device may also be for example a cell culture plate. The device may further also be any soft tissue contacting implant benefiting from tissue adhesion, improved healing of the wound, lack of encapsulation, faster growth of cells on the surface of the implant, lower inflammatory response and/or closing of the wound.

The present description also relates to a method for manufacturing a device capable of soft tissue adhesion, comprising the steps of
manufacturing a coating layer consisting essentially of titanium dioxide wherein
   at least 20% of the titanium dioxide has a crystalline structure of anatase and/or rutile, and
   the coating has a surface comprising indentations, wherein at least 50% of the indentations have a maximum depth of 1-50 nm and a maximum width of 1-50 nm,
treating the coating to achieve a water contact angle of 0-20°, and
treating the coating to be negatively charged or positively charged.

The treatment step can be carried out according to any of the methods disclosed above. Furthermore, all the other embodiments and details presented above in connection with the coating apply mutatis mutandis to the method.

The steps of manufacturing the coating layer and subjecting the coating layer to an excitation can be carried out either one after the other or almost simultaneously (while bearing in mind that the coating layer must exist before it can be treated).

According to an embodiment, manufacturing the coating layer is carried out using a sol-gel process, a hydrothermal process, a pulsed laser deposition process, an ion implantation process, an electrospraying process, a chemical vapour deposition process, a physical vapour deposition process, a laser beam machining process or a three dimensional growing process.

Without wishing to be bound to a theory, the Applicant believes that the oxidation reaction to decompose organic compounds is proceeded first by cutting molecular bonding to draw out hydrogen atoms and then adding oxygen atoms. In this process, when using UV light, the high energy UV radiation and radical oxygen atoms play an important role. A low pressure mercury lamp itself generates ozone and decomposes it to produce activated oxygen. It is an ideal light source for surface processing. It is believed that the 185 nm UV light decomposes oxygen molecules and synthesizes ozone 03. The 245 nm UV light decomposes ozone and produces high energy O* (activated oxygen). Thus radicals such as *OH, COO*, CO* and *COOH are formed with an increased hydrophilic nature. Organic molecules can be cracked and oxidized by UV light and active oxygen can be generated by UV light. CO₂ and H₂O are formed, which desorb from the surface. The surface will be thus be cleaned from organic contaminants and becomes hydrophilic. A photocatalytic surface creates a similar oxygen radical production by light photons below 360 nm. Thus it is not dependent on air molecules to turn into ozone first.

Plasma treatment of surfaces involves the removal of impurities and contaminants from surfaces through the use of an energetic plasma or dielectric barrier discharge (DBD) plasma created from gaseous species. Gases such as argon and oxygen, as well as mixtures such as air and hydrogen/nitrogen can be used. The plasma is created by using high frequency voltages (typically kHz to >MHz) to ionise the low pressure gas (typically around 1/1000 atmospheric pressure), although atmospheric pressure plasmas are also common. In plasma, gas atoms are excited to higher energy states and also ionized.

A plasma's activated species include atoms, molecules, ions, electrons, free radicals, metastables, and photons in the short wave ultraviolet (vacuum UV, or VUV for short) range. This “soup”, which incidentally is around room temperature, then interacts with any surface placed in the plasma. If the gas used is oxygen, the plasma is an effective, economical, environmentally safe method for critical cleaning. The VUV energy is very effective in the breaking of most organic bonds (i.e., C—H, C—C, C═C, C—O, and C—N) of surface contaminants. This helps to break apart high molecular weight contaminants. A second cleaning action is carried out by the oxygen species created in the plasma (O₂⁺, O₂⁻, O₃, O, O⁺, O⁻, ionised ozone, metastable excited oxygen, and free electrons). These species react with organic contaminants to form H₂O, CO, CO₂ and lower molecular weight hydrocarbons. These compounds have relatively high vapour pressures and are evacuated from the chamber during processing. The resulting surface is ultra-clean.

### EXPERIMENTAL PART

### Cell Culture Test Results

The present coating was tested as will be described below. The results of the cell culture tests show that the various material allow cell attachment and spreading onto their surfaces at levels that are comparable to each other. It shows that all tested materials are biocompatible and can be said to be good implant materials compared to other metals like surgical steel.

Differences become great when the cells are trypsinated under controlled protocol as explained below. The difference of the number of attached cells to number of cells left on the surface after trypsination can be taken as a measure of cell adhesion strength.

In this example it is shown that cell adhesion is increased with the three properties of the material surface. There are four materials of which two are essentially the same: sol-gel produced TiO₂ and hydrothermally produced TiO₂ both have nanostructure under the 50 nm limit and are crystalline in anatase phase. The third material is hydrothermally produced surface that has nanostructures above 50 nm while also being highly crystalline in anatase phase. The fourth material is machined titanium that has no anatase crystals and no nanostructure. However it has a naturally adsorbed extremely thin TiO₂ layer of about 3 nm thick.

Sol-gel produced or hydrothermal surfaces have thickness of typically above 200 nm and less than 1000 nm. The crystallinity of the sol-gel produced material is slightly lower than that of the hydrothermally produced material.

The test samples (six parallel samples) were either plate-like squares wherein each side was 8 mm long or discs with a diameter of 8 mm. The thickness of the samples were 0.4 mm. The results did not differ from squares to discs, as long as all the samples compared one to another had the same shape.

All materials were tested with and without UV cleaning. With UV cleaning the surface carbohydrates are removed and the surface undergoes also a photocatalytic reaction which turns it positively charged.

Test surfaces were treated in UV light chamber for 1 hour and kept in de-ionised water for 20 hours before cell culture test. A low pressure Hg fluorescent lamp, Osram Sylvania GFT36DI/2G11/SE/OF 36 W Compact UV Germicidal Lamp, measured to emit a light intensity of 3 W/m² at a distance of 2 mm from the sample, was used. The samples were measured to reach a water contact angle of 0-5 degrees during the treatment time. The storing in de-ionised water was carried out in room temperature, in a closed glass recipient.

The surfaces tested were as follows.

In the following, TiVAl stands for a blend of titanium-vanadium (4%) and aluminium (6%). This is a material commonly used for such abutments and can be also found as Grade 5 titanium.

Ceramic Zr stands for pure ZrO ceramic material obtained by cutting the green state ZrO block into shape using a diamond saw and sintering it according to manufacturers (Metoxit ag—Dental) recommendation at 1200° C. for 4 h.

Zr—Ti metallic alloy stands for a commercially available Roxolid® implant material produced by Institut Straumann Ag. It has approximately 8% of Zr in Titanium. The discs were cut out of a Zr—Ti rod of 8 mm diameter.

The following abbreviations are used.

TiVAl: a metallic titanium surface, without any treatment or coating, i.e. comparative material.

TiVAl UV: a metallic titanium surface, treated with ultraviolet light, i.e. comparative material.

Sol-gel Ti: a metallic titanium surface coated with a titanium dioxide coating having the crystalline structure and roughness as described here, made by sol-gel technique, i.e. comparative material.

Sol-gel Ti UV: the sol-gel surface as explained above, treated with ultraviolet light, i.e. according to the present invention.

HT>50 nm: a metallic titanium surface coated with a hydrothermally obtained titanium dioxide coating with a crystalline structure as described here but having a surface roughness with indentations of over 50 nm (both width and depth), i.e. comparative material.

HT<50 nm: as above, but with indentations below 50 nm, i.e. comparative material.

HT<50 nm UV: as above, additionally treated with ultraviolet light, i.e. according to the present invention.

Sol-gel Zr: a ceramic zirconium surface coated with a titanium dioxide coating having the crystalline structure and roughness as described here, made by sol-gel technique, i.e. comparative material.

Sol-gel Zr UV: the sol-gel surface as explained above, treated with ultraviolet light, i.e. according to the present invention.

HT Zr—Ti: a metallic alloy of Zr—Ti surface coated with a hydrothermally obtained titanium dioxide coating with a crystalline structure as described here and having a surface roughness with indentations of less than 50 nm (both width and depth), i.e. comparative material.

HT Zr—Ti UV: a metallic alloy of Zr—Ti surface coated with a hydrothermally obtained titanium dioxide coating with a crystalline structure as described here and having a surface roughness with indentations of less than 50 nm (both width and depth) and treated with ultraviolet light, i.e. comparative material.

Zr—Ti: a machined surface Zr—Ti metal alloy. The surface roughness of the machined surface was measure to be 0.1 Zr—Ti UV: a machined surface Zr—Ti metal alloy treated with ultraviolet light.

The materials used in the trypsination tests were as follows
Dulbecco's modified eagle medium (DMEM), 10% fetal calf serum (FCS)+penicillin+streptomycin (100 U/μg, Gibko BRL, Ufe technologies, Paisley UK)
Phosphate buffered saline (PBS)
Formalin solution, neutral buffered, 10%
0.25% trypsin-ethylene diamine tetra acetic acid (EDTA) diluted in the PBS to 0.0005 volume-% of trypsin in the PBS
Hoechst 33342-solution for staining
70% ethanol (Etax Aa, Altia Oy)

The coatings to be tested were first cleaned in a water containing ultrasonic bath. Firstly, each sample was put in a decanter containing acetone and cleaned for at least five minutes. Secondly, each sample was put in a decanter containing ethanol and cleaned for at least five minutes.

The cells used in the test were then passaged in cell culture flasks. The process was according to standard practice in the field, namely
warming up the growing medium and trypsin-EDTA in the bath,
removing the medium from the flask by pouring,
washing the cells twice with 20 ml PBS (in a 75 cm² flask),
detaching the cells with 5 ml of 0.25% trypsin-EDTA,
waving the bottle gently so that trypsin flows over the cells and after that pouring the trypsin away,
putting the flask in the incubator and waiting for the cells to detach (5-15 min),
preparing a new flask by adding 20 ml fresh medium (in a 75 cm² flask),
taking 9 ml of fresh medium in a glass pipette and flushing the cells a few times,
sucking the cells and medium in a pipette, and
adding a portion (typically 1:3) of the cells in the new prepared cell culture flask.

After passing, the cells were seeded on the samples to be tested. The samples were arranged on wells of a well place. The amount of cells was first measured with Bio-Rad cell counter (10 μl of coloured cell suspension on chip). Coloured cell suspension for chip was 20 μl colour+20 μl cells from medium. Amount of cells was marked as cells/ml. Thereafter, the amount of cells needed for each well was calculated and the correct amount of cells was mixed in fresh medium. A typical amount of cells was 10000 cells/cm², while the well surface area was 3.8 cm², thus resulting in approximately 38000 cells/well. The cell bottle was kept on a magnetic stirrer and mixed constantly. The desired amount of freshly made cell suspension was pipetted into the wells and the plates were incubated for 6 hours at 37° C.

Thereafter, the cell adhesion strength test phase (trypsinisation) was carried out. The test comprised the following steps
diluting trypsin-EDTA with PBS,
transferring samples in new fresh wells and filling with PBS,
washing the wells three times with PBS,
pipetting the diluted trypsin-EDTA on the samples,
placing the plates to an orbital shaker (50-100 rpm) and incubating for 10-15 min in 37° C.,
removing the trypsin-EDTA and pipetting DMEM in the wells,
washing the wells three times with PBS, and
fixing the cells without delay.

The cells were fixed using the following procedure.
removing the PBS from the wells,
fixing the cells with formalin by pipetting formalin into the wells,
waiting for 15 min,
washing the wells twice with PBS, and
leaving the PBS in the wells.

Cells were either stained right after the fixing, or the wells were placed in the fridge and stained later. The well plates were then prepared for imaging, for studying the adhesion of the cells. This step could also be done later or right after the fixation, with no effect to the results.

The staining of the cells consisted of the following steps.
preparing a dilution of the Hoechst 33342-solution,
removing the PBS,
pipetting the Hoechst 33342-solution in the wells,
placing plates to the orbital shaker (50-100 rpm) and incubating for 15 min,
washing wells three times with PBS,
leaving PBS in the wells,
wrapping the plates in foil to protect the stain from light, and
imaging plates.

The imaging of the plates was carried out as follows: The cells were stained with Hoechst 33342 nucleid acid stain and the non-trypsinated and trypsinated samples were imaged with a fluorescence microscope. Cells in the non-trypsinated and trypsinated image were counted and a detachment percentage was calculated by comparing the number of cells at trypsinated sample to the number of cells in the non-trypsinated sample.

Some samples of hydrothermally coated TiVAl were subjected to a relative activity measurement, in order to verify that the coatings were indeed photocatalytically active. The activity was measured as the materials ability to degrade methylene blue (MB) from water solution under UV irradiation. The products used were methylene blue, C₁₆H₁₈N₃SCl (Merck, 1.59270.0010) and purified water (resistivity 18.2 MΩcm). In the measurement, the samples were placed on the bottom of 4 ml cuvettes and 2 ml of operating solution (1*10⁻⁵ M) was pipetted on the samples. The cuvettes with samples and operating solutions were then stabilised in dark and after one hour, the “zero value” was determined by UV-Vis spectrophotometer at the wavelength of 660 nm.

After determination the absorbance of the “zero value” the samples with solutions were subjected to UV irradiation in a reaction chamber. The cycle was repeated with one hour time interval for a total of 5 hours i.e. the samples were irradiated for a total 5 hours with UV light. The UV-lamps were located 5-6 cm above the samples, and spectrophotometer measurements were carried out. The results were calculated by converting the measured relative absorbance changes to logarithmic scale and calculating the linear coefficient (slope) and correlation factor R (correlation or Pearson). At least three measurement points were used. Thereafter, the slope value was multiplied by factor 1000 and by used volume (ml) of operating solution and divided by sample's surface area (area which is under UV radiation) and by number 7 (scale factor). The results are given as the relative kinetic constant vs. surface area (Relative activity/cm²).

The results are shown in Table 1 below, and all shown that the samples were photocatalytically active.

TABLE 1

| | | | area of | | |
|---|---|---|---|---|---|
| | | | sample (A) | Slope/ | Relative |
| Sample | R | Slope | [cm²] | Area | activity × 10³ |
| 1 | 0.99562 | 0.03990 | 0.7800 | 0.0512 | 51.15 |
| 2 | 0.995097 | 0.04456 | 0.8260 | 0.0539 | 53.94 |
| 3 | 0.998239 | 0.03295 | 0.7720 | 0.0427 | 42.68 |
| 4 | 0.992839 | 0.0325143 | 0.7770 | 0.0418 | 41.85 |
| 5 | 0.998247 | 0.03197 | 0.7180 | 0.0445 | 44.53 |
| 6 | 0.997141 | 0.03284 | 0.7510 | 0.0437 | 43.73 |

Some samples of hydrothermally coated TiVAl were also tested with X-ray photoelectron spectroscopy (XPS) to estimate the amount of carbon atoms on the surface of the coated samples. The results showed that indeed, UV-treatment diminished the amount of carbon atoms on the surface, and the effect was dependent on the treatment time. Results are given below in Table 2, wherein “water” means the sample was washed with water, “ethanol” means the sample was washed with ethanol, “before UV” means a non-washed sample and the three remaining results illustrate various lengths of UV-treatment. The samples were tested immediately after their coating.

TABLE 2

| | C | O | Ti |
|---|---|---|---|
| Water | 17.8 | 62.2 | 14.9 |
| Ethanol | 11.7 | 65.7 | 16.7 |
| Before UV | 24.5 | 57.0 | 13.3 |
| 5 min UV | 18.5 | 60.3 | 14.5 |
| 15 min UV | 13.7 | 64.4 | 16.0 |
| 60 min UV | 8.5 | 68.3 | 17.0 |

### In Vitro Test Results

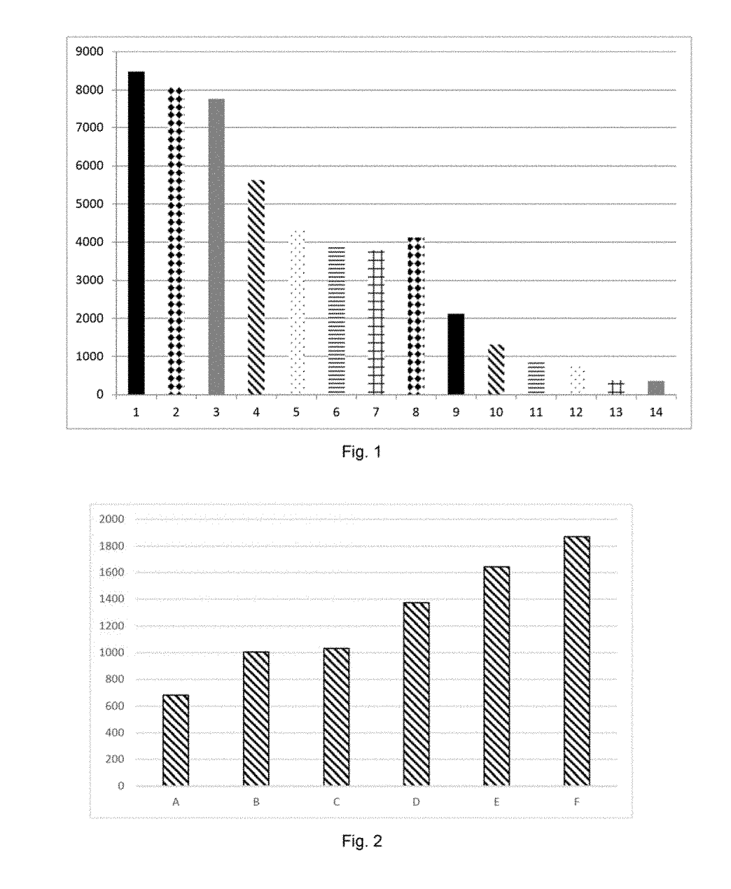

Some test results are given in FIG. 1, where cell counts were taken both before and after (adh) trypsination.

The column in FIG. 1 represent the samples as follows (explanation of the abbreviations above). Column 1 stands for HT<50 nm, Column 2 stands for HT<50 nm UV; Column 3 stands for HT>50 nm; Column 4 stands for sol-gel Ti UV; Column 5 stands for sol-gel Ti; Column 6 stands for TiVAl UV, Column 7 stands for TiVAl; Column 8 stands for HT<50 nm UV adh; Column 9 stands for HT<50 nm adh; Column 10 stands for sol-gel UV adh; Column 11 stands for TiVAl UV adh; Column 12 stands for sol-gel adh; Column 13 stands for TiVAl adh; Column 14 stands for HT>50 nm adh.

The order of number of attached cells after incubation and before trypsination, from lower number to higher number, was:

TiVAl, TiVAl UV, sol-gel, sol-gel UV, HT>50 nm, HT<50 UV, HT<50.

The order of cell adhesion strength, i.e. the number of attached cells after trypsination, was (from lower to higher number):

HT>50 nm, TiVAl, sol-gel, TiVAl UV, sol-gel UV, HT<50 nm, HT<50 UV.

It can thus be seen that the surface roughness under 50 nm according to the present invention Improves cell adhesion, as does treatment with UV.

Indeed, comparison of the test materials revealed that

1. HT<50 nm vs. HT>50 nm: although both allow cell attachment and spreading very well, only HT<50 nm allows also adherence of cells.
2. HT<50 nm vs. HT<50 nm UV: both attached and spread cells very well, but UV treatment increased adhesion significantly
3. All materials vs. all materials with UV: UV increases adhesion of cells. Different substrates benefit in different degree from the UV treatment.
4. All coated vs. non-coated: all coated materials had increased attachment and adhesion vs. non coated. HT>50 nm was an exception showing a very low adhesion of cells. Different substrates benefit in different degree from the coating.
5. HT<50 nm UV vs all others: the combination of the nanostructure<50 nm, highly crystalline anatase crystals and UV gives the greatest adhesion of all.

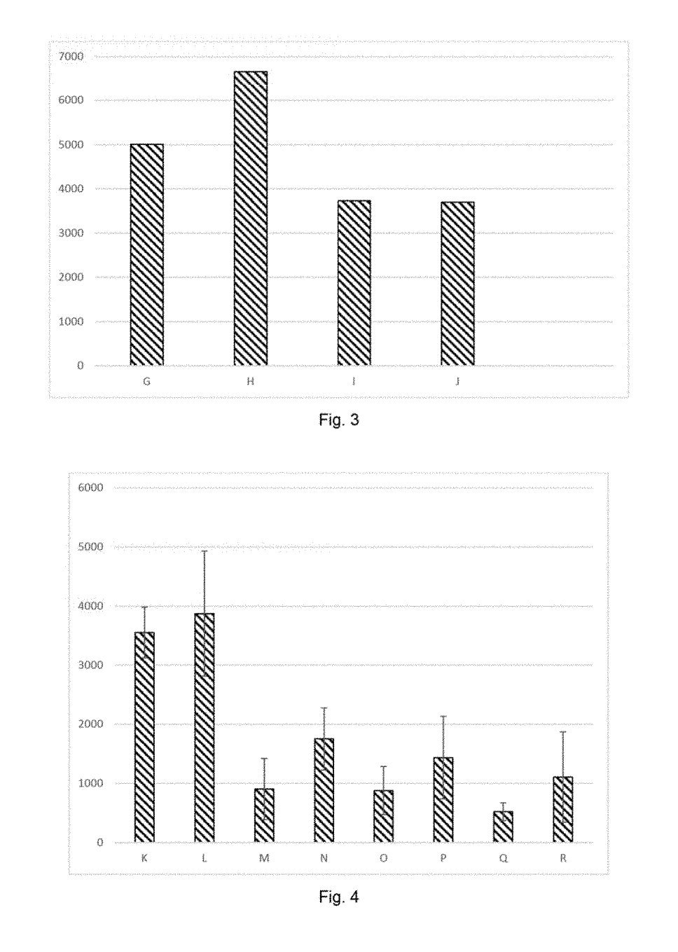

FIGS. 2 and 3 show results of trypsination test, i.e. the amount of cells that remained attached after the trypsinations. In FIG. 2, the substrate used was titanium, and the results are for A non-coated non-UV-treated, B non-coated UV-treated, C sol-gel coated non-UV-treated, D sol-gel coated UV-treated, E hydrothermally coated non-UV-treated and F hydrothermally coated UV-treated.

In FIG. 3, the substrate used was zirconium, and the results are for G sol-gel coated non-UV-treated, H sol-gel coated UV-treated, I non-coated non-UV-treated and J non-coated UV-treated.

In FIG. 4, the substrate was either the alloy of zirconium and titanium (Zr—Ti), and the coating was made with hydrothermal treatment, or the substrate was TiVAl and the coating was made with sol-gel process. In the Figure, K stands for Zr-TI non-UV-treated; L for Zr—Ti UV-treated, M for TiVAl non-UV-treated, N for TiVAl UV-treated, O for non-coated TiVAl non-UV-treated, P for non-coated TiVAl UV-treated, Q for non-coated Zr—Ti non-UV-treated and N for non-coated Zr—Ti UV-treated. Some samples of TiVAl were tested with a blood coagulation (or blood clotting) method. Indeed, the thrombogenicity of the bioactive surfaces was evaluated using a whole blood kinetic clotting time method, as previously described (Imai & Nose, 1972; Huang et al., 2003). Fresh human whole blood was drawn from a healthy adult volunteer who had not taken any medication affecting platelets function, by venipuncture into vacutainer tubes. The first 3 ml of the drawn blood was discarded to avoid contamination by tissue thromboplastin caused by needle puncture. Briefly, a 100 μl volume of blood was carefully added to the surfaces (n=4), which were placed into 12-well plates. All substrates were incubated at room temperature for 10, 20, 30, 40, 50, and 60 min. At the end of each time point, the substrates were incubated for 5 min with 3 ml of ultrapure water. Each well was sampled in triplicate (200 μl each) and transferred to a 96-well plate. The red blood cells that were not trapped in a thrombus were lysed with the addition of ultrapure water, thereby releasing hemoglobin into the water for subsequent measurement. The concentration of hemoglobin in solution was assessed by measuring the absorbance at 570 nm using ELISA plate reader. The size of the clot is inversely proportional to the absorbance value.

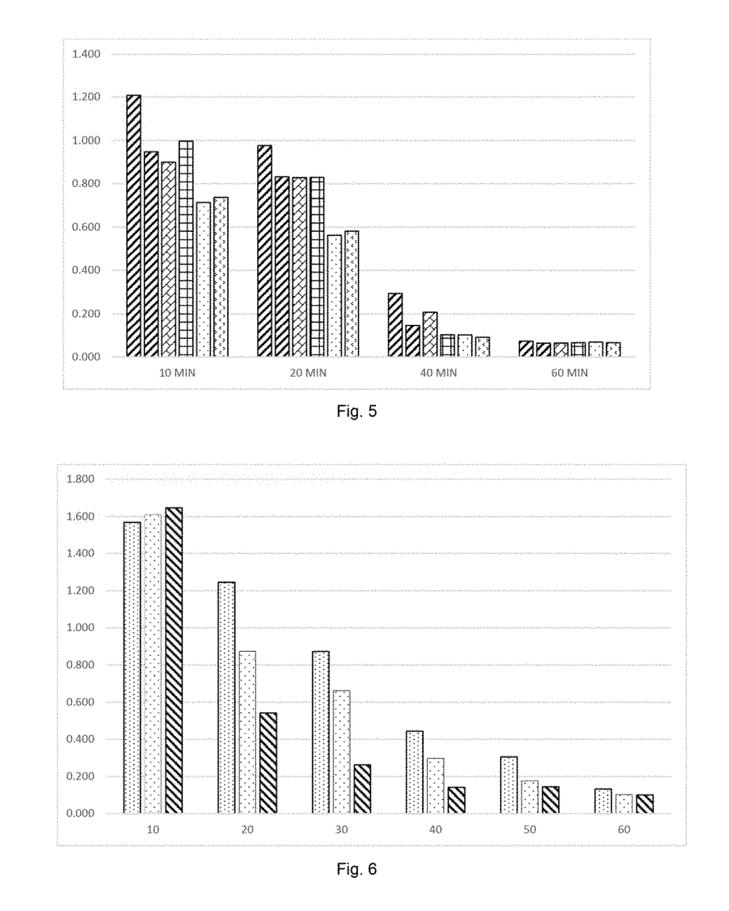

The results are shown in FIG. 5, at 10, 20, 40 and 60 minutes, time vs. absorbance. The samples that were tested are, in each group from left to right (columns) non-UV-treated and non-coated; non-UV-treated sol-gel coated; non-UV-treated hydrothermally coated; UV-treated non-coated; UV-treated sol-gel coated and UV-treated hydrothermally coated. As can be seen, UV-treatment has an effect on each type of sample (coated or non-coated), and the coating according to the present description has the greatest effect on decrease of the absorbance, i.e. Increase in blood coagulation. Increased blood coagulation predicts better cell adherence.

Some zirconium-samples were also tested with the coagulation test as described above. These results are shown in FIG. 6, at 10, 20, 30, 40, 50 and 60 minutes, time vs. absorbance. The samples that were tested are, in each group from left to right (columns) non-UV-treated and non-coated, non-UV-treated sol-gel coated and UV-treated sol-gel coated. The results are similar to those with TiVAl.

A still further test was carried out, as it was observed that in the cell adhesion tests, a number of effects were discovered that distorted the results with respect to the observed clinical outcomes and between individual cell culture test rounds. Without wishing to be bound to a theory, the present inventors believe the following.

Carrying out a cell culture test with a non-coated titanium surface had the most stable outcome from test to test. This can be understood since such surface has the least reactive sites and the surface is already occupied with naturally occurring carbon compounds directly from the air as demonstrated in various publications.

A UV-cleaned surface typically increased the cell adhesion by a variable amount, which difference however was mostly within the standard deviation of the two groups. The standard deviation can be understood to be necessarily big compared to actual values since cell cultures are difficult to standardise due to the biological factors in the test. However a large enough and statistically significant difference can be observed for all groups.

One result-distorting factor emerged also in the “sol-gel coated and UV treated” as well as “HT-coated and UV treated” groups. It was observed that if the samples were placed directly in a cell culture after UV treatment, the UV treated samples had a very high standard deviation compared to untreated coated samples and control samples. In addition, their actual values could vary from one cell culture to another tremendously and not in a logical manner. This effect was discovered to be caused by a photocatalytic decay phenomenon, which has been mentioned in publications and assumed to exist for approximately 20 h after the UV-light is switched off. In practise the anatase containing surface continue to store electron holes after UV-treatment and release them in the next hours, continuously creating oxygen radicals, which in turn attack all organic matter on the surface of the material.

Therefore, it was concluded that the cell culture should only be performed after 20 hours of the UV-treatment, i.e. after the decay was mostly over. Thus a cell culture test was performed by placing all samples in deionised water 20 hours prior to the cell adhesion test.

With this test setup, the variation of the coated and UV-treated groups disappeared as was expected.

However, the difference between the actual values of the coated and coated+UV-treated groups became smaller than earlier and what was expected based on the clinical results. It was concluded that the storage in deionised water kept the UV-treated surface clean, increasing the cell adhesion compared to coated but untreated group, as expected according to the invention. However, an effect seemed to be missing from the expected.

The next step toward a clinically comparable situation was to add proteins into the test. At the same time it was necessary to avoid the detrimental effect of the photocatalytic effect. The photocatalytic decay effect per se is small in quantity, but big enough to have an effect at cell culture volumes of biomass. However, in a clinical situation, the surrounding biomass and fluid flow is thousands of times higher. Thus the detrimental effect cannot be observed in a clinical situation. Secondly, the half time of the decay is 6 hours. Thus most of the effect is gone already after 12 hours. The cell adhesion test takes 6 hours to allow cells to adhere after the liquid volume is suspended on the sample. Thus in a normal cell culture, setting the adhering cells would receive also 50% of the photocatalytic decay effect.

Thereafter, a serum modified test was carried out. The serum was DMEM (Dulbecco's Modified Eagle's Medium), which is a 10% diluted serum, containing thus a fairly low concentration of proteins. The samples were stored in closed glass vials for 20 hours in darkness, to prevent further photocatalysis.

Samples of coated and coated+UV treated groups as well as controls, were placed in serum, containing all necessary proteins for cell viability and adhesion, for 20 hours before the cell adhesion test. Thus proteins were allowed to adsorb to the surface while the decay was ongoing. As soon as an electron hole had decayed to its original state, released its energy, created an oxygen radical, which had broken the nearby organic compound, the cleaned position on the surface was be occupied by a new serum protein. This mimics closely also the clinical situation where the surface would come into contact with blood and body fluids nearly immediately after the UV treatment.

Thus, if the coated and UV cleaned surface has more available sites for protein adsorption than just coated or non-coated, then this should be seen in the amount of cell adhesion.

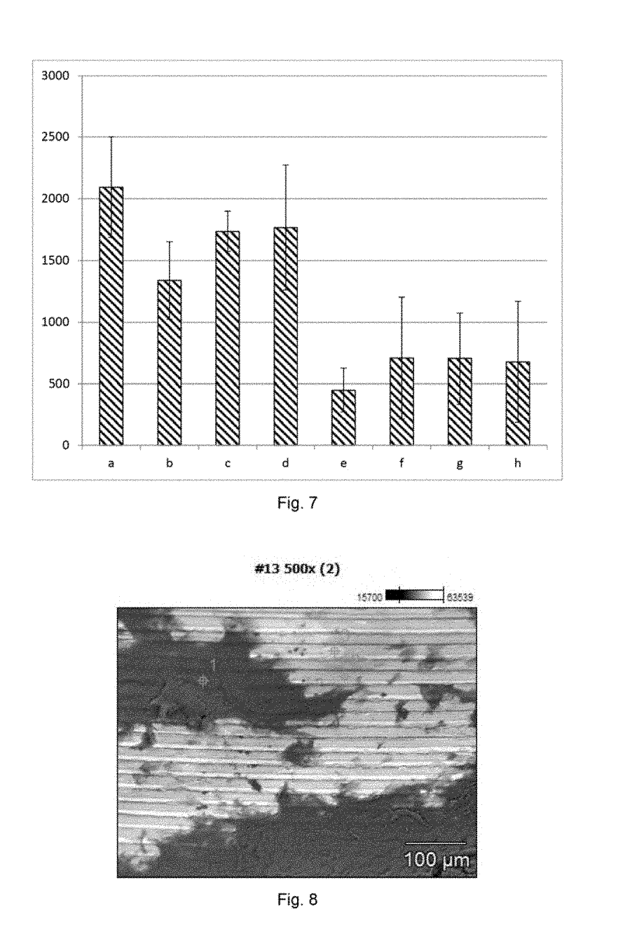

The results were as follows, and are also illustrated in FIG. 7. The storage time was 20 hours in each case. In FIG. 7, the columns illustrate the results of the following groups: a stands for HT-coated, UV-treated samples stored in serum; b stands for HT-coated, non-UV-treated samples stored in serum; c stands for HT-coated, UV-treated samples stored in water; d stands for HT-coated, hydrogen peroxide-treated samples stored in serum; e stands for non-coated, hydrogen peroxide-treated samples stored in serum; f stands for non-coated, UV-treated samples stored in serum; g stands for non-coated, non-UV-treated samples stored in serum; h stands for non-coated, non-UV-treated samples stored in water.

The samples could be grouped as follows, from best to worst group.

Group 1: the best samples were the HT-coated and UV-treated, stored in serum (a).
Group 2: the two next best samples were the HT-coated, UV treated, stored in water (c) and the HT-coated, hydrogen peroxide-treated and stored in serum (d).
Group 3: the next best samples were the HT-coated, no UV, stored in serum (b).
Group 4: the non-coated samples (e-h) exhibited low cell adhesion values compared to the coated ones, regardless of the cleaning method or storage as expected.

All results are thus as expected according to the present disclosure.

Particularly the difference between groups 3 and 2 is evidence of the effect of cleaning, i.e. lowering the water contact angle. Despite group 3 being stored in serum, it shows a lower affinity to cells than the same surface after cleaning in UV and storing in water, or cleaning in H₂O₂ and storing in serum.

### In Vivo Test Results

The present invention was tested in a clinical procedure, using dental healing abutments. The healing abutments were commercially available DESS-abutments from Spain.

### Material Treatment

The healing abutments were divided into groups that were treated in different manners.

Seven abutments were coated with a sol-gel processed nanoporous surface, containing mainly of crystalline anatase phase. The TiO₂ sol was prepared to produce the coatings by using the dip-coating procedure. The TiO₂ sol was prepared by dissolving tetraisopropylorthotitanate (TIPT, Ti((CH₃)₂CHO)₄) in absolute ethanol and mixing with a solution containing ethyleneglycolmonoethylether (C₂H₅OCH₂OH), deionized water, fuming hydrochloric acid (HCl, 37%) and ethanol. The sol was aged 24 h at 0° C. before dipping. The abutments were cleaned in an ultrasonic bath using acetone and alcohol for 5 min each. After drying the abutments were dip coated in the sol. Subsequently the abutments were heat treated at 500° C. for 1 h. After heat treatment the abutments were washed again in acetone and alcohol ultrasonically for 5 min.

Four abutments were non-coated as machined TiVAl abutments.

All abutments were sterilized by autoclaving prior to a final surface cleaning treatments and implantation.

Following final treatments were performed and the following abutments tested:
three sol-gel coated abutments without further cleaning
two sol-gel coated and UV treated abutments
two sol-gel coated and plasma treated abutments
two UV treated, non-coated abutments
two plasma treated, non-coated abutments.

The surfaces were treated (when applicable, see above) using Superosseo ST-24066 UV light oven (Ushio, Japan) in a 12 min cleaning cycle, or using Yocto type Argon plasma cleaner (Diener Electronic GmbH+Co. KG, Ebhausen, Germany) in a 11 min 40 s cycle, prior to implantation.

### Implantation

At the day of surgery a dental implant was placed into the jawbone of the patient. Following this procedure a dental implant healing abutment was placed on top of the implant by screwing it in place, penetrating the gingival tissue. Thus a part of the healing abutment was inside the implant, another part was in contact with the gingival tissue and the top face was in the oral cavity, free of tissue contact. The wound was sutured closed and left in close contact to heal over a period of at least 12 weeks.

### Follow Up Method

The healing abutments were followed clinically for 12 to 18 weeks after which they were removed.

The healing abutments were removed by unscrewing them anticlockwise.

The removed abutment was placed in a formaldehyde fixing solution for the transport period. The samples were dried and the amount of tissue covering the surfaces were studied under a scanning electron microscope.

The measurement of the tissue covering the contact area was done from the image using image analysis. The tissue edge was drawn on the image and compared to the total area defined. The limits of the contact area were defined as the implant-abutment interface and the top corner of the healing abutment, although tissue may have been covering also areas above the top corner.

### SEM-Images

In total nine samples were analysed for the tissue coverage, according to Table 3.

TABLE 3

| Sample | Tissue coverage % |
|---|---|
| Sol-gel coated | 28.4 |
| Sol-gel coated | 49.3 |
| Sol-gel coated, UV treated | 67.5 |
| Sol-gel coated, UV treated | 85.5 |
| Sol-gel coated, plasma treated | 69.4 |
| Sol-gel coated, plasma treated | 39.8 |
| Machined, UV treated | 16.9 |
| Machined, UV treated | 40.3 |
| Machined, plasma treated | 0 |
| Machined, plasma treated | 43.7 |

The average tissue coverage was 38.8% for sol-gel coated surface (i.e. not according to the present description), 65.5% for sol-gel coated and UV or plasma treated surface and 25.5% for UV or plasma treated titanium (i.e. not according to the present description). In addition the rupture with the sol-gel coated and UV or plasma treated surfaces occurred clearly within the tissue, whereas with sol-gel or UV or plasma treatment alone the rupture occurred partially within the tissue and partially at the interface of the protein layer and tissue as well as at the abutment interface. It is to be noted that this test is different from the trypsination test mentioned above.

These results indicate that the strength of tissue adhesion is greatest with the nanostructured, crystalline and UV or plasma treated surfaces. Clinically the most significant outcome is that the gingival tissue would have to rupture in order for the abutment to loose contact with the tissue. With sol-gel coated or plasma or UV treated surfaces, the loss of contact can be partially achieved without rupture of the tissue, leading to the existence of a gate for bacteria to enter. Thus this difference in tissue adhesion strength has a very profound effect for the clinical outcome.

### Elemental Analysis

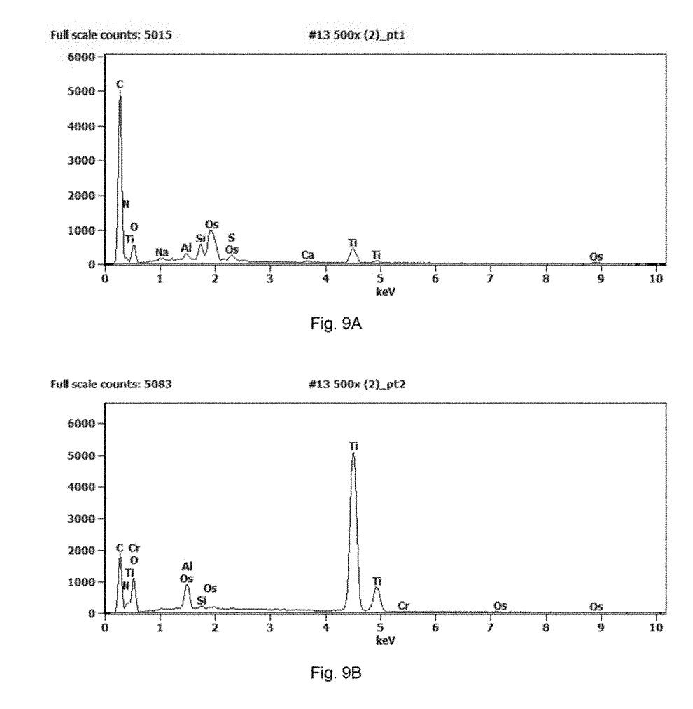

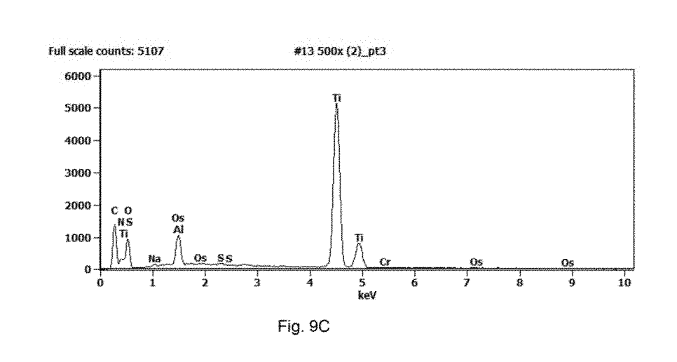

Surface areas that had been uncovered of tissue structures had remnants of thin organic matter on them. In the elemental analysis carbon was shown to have increased levels compared to other areas where visibly no organic layers were detected. This was interpreted as protein adsorption and adhesion on the coated surfaces. The surface area covered by tissue was interpreted as a clear layer of organic matter on top of the oxide or metal surface. FIG. 8 shows a microscopic image of one tested surface, where the different areas can be seen and FIGS. 9A-9C show the elemental analysis at three different points of FIG. 8, as indicated in FIG. 8.

1. A coating consisting essentially of titanium dioxide wherein
at least 20% of the titanium dioxide has a crystalline structure of anatase and/or rutile,
the coating has a surface comprising indentations, wherein at least 50% of the indentations have a maximum depth of 1-45 nm and a maximum width of 1-50 nm,
the coating is treated to achieve a water contact angle of 0-20° by at least one of
cleaning the surface of naturally adsorbed hydrocarbons by hydrogen peroxide or calcium peroxide, or by adsorbing calcium on the surface,
cleaning the carbohydrates and photocatalytically exciting the surface with UV light under 360 nm or by argon ion plasma or by oxygen plasma, and
the treated coating exhibits an attachment of mammalian tissue cells capable of adhesion, which attachment is such that when a substrate coated with the above coating and treated is compared to the same uncoated and untreated substrate, at least 100% more cells remain attached on the coated, treated substrate than on the uncoated, untreated substrate, the attachment of cells being measured by a method in which
six parallel plate-like samples of both coated, treated substrate and uncoated, uncoated substrate having a largest diameter of 8 mm and a thickness of 0.1-1 mm are used,
the samples are stored in an aqueous solution for at least 20 hours prior to the measurement,
soft tissue cells are cultured on the coated, treated substrate samples and uncoated, untreated substrate samples for 6 hours at 37° C. in a 5% carbon dioxide atmosphere,
the cultured coated, treated substrate samples and the cultured uncoated, untreated substrate samples are subjected to trypsinisation with a solution of trypsin in phosphate buffered saline in an orbital shaker at 50 rpm for 12 min at room temperature, and
remaining attached cells are counted from the samples and the average of the six parallel samples is calculated,
wherein the volume-% of trypsin in the solution of trypsin is selected such that on average at least 300 cells/cm² remain attached on the uncoated, untreated substrate samples.

2. A coating according to claim 1, characterised in that it has been manufactured using a sol-gel process, a hydrothermal process, a pulsed laser deposition process, an ion implantation process, an electrospraying process, a chemical vapour deposition process, a physical vapour deposition process, a laser beam machining process or a three dimensional growing process.

3. (canceled)

4. A coating according to claim 1, wherein the water contact angle is 0-10°.

5. A coating according to claim 1, wherein at least 60% of the indentations have a maximum depth of 1-45 nm and a maximum width of 1-50 nm.

6. (canceled)

7. A coating according to claim 1, wherein the volume-% of trypsin in the solution of trypsin is selected such that on average at least 1000 cells/cm² remain attached on the uncoated, untreated substrate samples.

8. A coating according to claim 1, wherein the coating is treated to be negatively charged or positively charged.

9. A coating according to claim 1, wherein the aqueous solution is selected from deionised water and an aqueous solution comprising serum.

10. Use of a coating according to claim 1, for improving mammalian tissue cell adhesion of a device.

11. A medical or cosmetic device intended to penetrate a skin or to be implanted into a mammal, comprising a coating according to claim 1.

12. A device according to claim 11, characterised in that the device is selected from a group consisting of orthopaedic fixation pins, craniomaxillofacial prosthesis fixation screws, catheters, cannulae, vascular stents, aneurysm stents, esophageal stents, annular rings for heart valves, removable prosthesis implants for extremities, abdominal catheters, urethral catheters, dental implant abutments, tissue level implants abutment sections and skin penetrating jewelry.

13. A method for manufacturing a device capable of soft tissue adhesion, comprising the steps of
manufacturing a coating layer consisting essentially of titanium dioxide wherein
at least 20% of the titanium dioxide has a crystalline structure of anatase and/or rutile,
the coating has a surface comprising indentations, wherein at least 50% of the indentations have a maximum depth of 1-45 nm and a maximum width of 1-50 nm, and
treating the coating to achieve a water contact angle of 0-20° by at least one of
cleaning the surface of naturally adsorbed hydrocarbons by hydrogen peroxide or calcium peroxide, or by adsorbing calcium on the surface,
cleaning the carbohydrates and photocatalytically exciting the surface with UV light under 360 nm or by argon ion plasma or by oxygen plasma,
treating the coating to be negatively charged or positively charged.

14. A method according to claim 13, characterised in that manufacturing the coating layer is carried out using a sol-gel process, a hydrothermal process, a pulsed laser deposition process, an ion implantation process, an electrospraying process, a chemical vapour deposition process, a physical vapour deposition process, a laser beam machining process or a three dimensional growing process.
